(19) United States
(12) Patent Application Publication
(10) Pub. No.: US 2015/0258254 A1
(43) Pub. Date: Sep. 17, 2015

(54) METHODS FOR IN VIVO AND IN VITRO USE OF GRAPHENE AND OTHER TWO-DIMENSIONAL MATERIALS

(71) Applicant: LOCKHEED MARTIN CORPORATION, Bethesda, MD (US)

(72) Inventors: Sarah SIMON, Baltimore, MD (US); John B. STETSON, JR., New Hope, PA (US)

(21) Appl. No.: 14/656,190

(22) Filed: Mar. 12, 2015

Related U.S. Application Data
(60) Provisional application No. 61/951,926, filed on Mar. 12, 2014.

(51) Int. Cl. A61L 31/12; A61L 31/16

(52) U.S. Cl. CPC A61L 31/122; A61L 31/16; A61L 2300/252

(57) ABSTRACT

Two-dimensional materials, particularly graphene-based materials, having a plurality of apertures thereon can be formed into enclosures for various substances and introduced to an environment, particularly a biological environment (in vivo or in vitro). One or more selected substances can be released into the environment, one or more selected substances from the environment can enter the enclosure, one or more selected substances from the environment can be prevented from entering the enclosure, one or more selected substances can be retained within the enclosure, or combinations thereof. The enclosure can for example allow a sense-response paradigm to be realized. The enclosure can for example provide immunoisolation for materials, such as living cells, retained therein.

### CROSS-REFERENCE TO RELATED APPLICATIONS

This application claims the benefit of U.S. provisional application 61/951,926 filed Mar. 12, 2014 which is incorporated by reference herein in its entirety.

### BACKGROUND OF THE INVENTION

The present disclosure generally relates to transportation and delivery of substances in a biological environment, and, more specifically, to methods and devices for transportation and delivery of substances using a carbon nanomaterial.

Drug and cell delivery in both immune competent and immune incompetent organisms is a real and current problem in medical research and practice today. Present studies use polymeric devices and hydrogels as a delivery vehicle. Some examples include polytetrafluoroethylene with a backing of unwoven polyester mesh, silicon, hydrogels, alginate. cellulose sulfate, collagen, gelatin, agarose, chitosan and the like. Current delivery vehicles and devices are challenged by biofouling, biocompatibility issues, and delayed response. The thickness of current state devices can limit efficacy as limited diffusion of nutrients can kill cells contained within, or delay bi-directional transport of drugs or molecules that are being sensed. Low permeability, at least in part, due to thickness and mechanical stability in view of physical stress and osmotic stress can also be problematic.

In view of the foregoing, improved techniques for transportation and delivery of substances under a variety of conditions, particularly in a biological environment, would be of considerable benefit in the art. The present disclosure satisfies this need and provides related advantages as well.

### SUMMARY OF THE INVENTION

The present disclosure describes enclosures formed from perforated graphene or other perforated two-dimensional materials. The enclosures can house various substances therein allowing bi-directional movement of selected substances to and from the interior of the enclosure, retaining other selected substances therein and preventing entry of yet other selected substances into the enclosure. The enclosure of the invention can be employed to release one or more selected substances into an environment external to the enclosure, to allow entry into the enclosure of one or more selected substances from an environment external to the enclosure, to inhibit and preferably prevent entry of one or more selected substances from the external environment into the enclosure, to retain (inhibit or preferably prevent exit) one or more selected substances within the enclosure or a combination of these applications. The hole or aperture size or range of sizes is selected based on the specific application of the enclosure. The term enclosure refer to a space for receiving one or more substances formed at least in part by perforated two-dimensional material, such as a graphene-based material, where one or more substances in the enclosure can exit the enclosure by passage through the perforated two-dimensional material. Similarly, in certain embodiments, one or more substances from the external environment can enter the enclosure by passage through the perforated two-dimensional material. In specific embodiments the external environment is a biological environment, which may be an in vivo biological environment or an in vitro biological environment.

In embodiments, an enclosure comprises one or more than one sub-compartments each sub-compartment comprising perforated two-dimensional material such that at least a portion of the walls or sides forming the sub-compartment are perforated two-dimensional material. Fluid communication is achieved by selective passage of one or more substance in and/or out of the enclosure or sub-compartment thereof. The fluid may be liquid or gas and includes fluids having entrained gases. Substances may be dissolved or suspended or otherwise carried in a fluid. The fluid can be aqueous. A sub-compartment can be in direct fluid communication with adjacent sub-compartments or the external environment (where adjacent sub-compartments share at least one wall or side). In an embodiment one or more sub-compartments can be in direct fluid communication with adjacent sub-compartments, but not in direct fluid communication with the external environment. At least one sub-compartment in an enclosure is in direct fluid communication with an external environment. An enclosure can have various configurations of sub-compartments. A sub-compartment can have any shape. A sub-compartment may, for example, be spherical, cylindrical or rectilinear In an embodiment, sub-compartments can be nested. In an embodiment, the enclosure can have a central sub-compartment which shares a wall or side with a plurality of surrounding sub-compartments. In an embodiment, sub-compartments may be linearly aligned within the enclosure. In an embodiment, an enclosure contains two-sub-compartments. In an embodiment, an enclosure contains three, four, five or six sub-compartments. In an embodiment, a sub-compartment may be fully contained within another sub-compartment, wherein the inner sub-compartment is in direct fluid communication with the outer sub-compartment and the outer-sub-compartment is in direct fluid communication with the external environment. In this embodiment, the inner sub-compartment is in indirect rather than direct fluid communication with the external environment. In an embodiment where an enclosure contains a plurality of sub-compartments, at least one sub-compartment is in direct fluid communication with the external environment and remaining sub-compartments are in direct fluid communication with adjacent sub-compartment, but may not all be in direct fluid communication with the external environment. In an embodiment where an enclosure contains a plurality of sub-compartments, all sub-compartments may be in direct fluid communication with the external environment.

An enclosure encapsulates at least one substance. In an embodiment, an enclosure can contain more than one different substance. Different substances may be in same or in different sub-compartments. In an embodiment, not all of the different substances in the enclosure are released to an environment external to the enclosure. In an embodiment, all of the different substances in the enclosure are released to an external environment. In embodiments, the rate of release of different substances from the enclosure into an external environment is the same. In embodiments, the rate of release of different substances from the enclosure into an external environment is different. In an embodiment, the relative amounts of different substances released from the enclosure can be the same or different. The rate of release of substances from the enclosure can be controlled by choice of hole size, hole functionalization or both.

Methods for transporting and delivering substances in a biological environment are also described herein. In some embodiments, the methods can include introducing an enclosure formed from graphene or other two-dimensional material into a biological environment, and releasing at least a portion of a substance in the enclosure to the biological environment. In some or other embodiments, the methods can include introducing an enclosure formed from graphene into a biological environment, and migrating a substance from the biological environment into the enclosure.

In an embodiment, the invention provides a method comprising:

introducing an enclosure comprising perforated two-dimensional material to a an environment, the enclosure containing at least one substance; and

releasing at least a portion of at least one substance through the holes of the two-dimensional material to the environment external to the enclosure. Any enclosure herein can be employed in this method.

In an embodiment, the invention provides a method comprising:

introducing an enclosure comprising perforated two-dimensional material to a environment, the enclosure containing at least one first substance; and migrating a second substance from the environment into the enclosure. In an embodiment, the first substance is cells, a second substance is nutrients and another second substance is oxygen.

The foregoing has outlined rather broadly the features of the present disclosure in order that the detailed description that follows can be better understood. Additional features and advantages of the disclosure will be described hereinafter. These and other advantages and features will become more apparent from the following description.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 shows an illustrative schematic demonstrating the thickness of graphene-based material in comparison to conventional drug delivery vehicles and devices. This figure also illustrates an embodiment of the invention in a biological environment in contact with biological tissue in which enclosure is provided with one or more support materials which re external to the perforated two-dimensional material and indicates possible capillary vascularization into such support materials.

FIGS. 2A-D shows illustrative schematics of various configurations of enclosure configurations prepared from two-dimensional material useful according to various embodiments of the present disclosure.

FIGS. 3A and 3B are schematic illustrations of an enclosure of the invention implemented for immunoisolation of living cells.

FIGS. 4A-C illustrate exemplary preparation of an enclosure of the invention.

### DETAILED DESCRIPTION OF THE INVENTION

The present disclosure is directed, in part, to methods for using graphene-based materials and other two-dimensional materials to transport and deliver substances in a biological environment. The present disclosure is also directed, in part, to enclosures formed from graphene-based materials and other two-dimensional materials on or suspended across a suitable substrate or substrates which can be porous or non-porous, which can serve as a delivery vehicle in an environment external to the enclosure, particularly in a biological environment. The present disclosure is also directed, in part, to enclosures containing cells, pharmaceuticals and other medicaments formed from graphene-based materials or other two-dimensional materials.

Graphene has garnered widespread interest for use in a number of applications due to its favorable mechanical and electronic properties. Graphene represents an atomically thin layer of carbon in which the carbon atoms reside as closely spaced atoms at regular lattice positions. The regular lattice positions can have a plurality of defects present therein, which can occur natively or be intentionally introduced to the graphene basal plane. Such defects will also be equivalently referred to herein as “apertures,” “perforations.” or “holes.” The term “perforated graphene” is used herein to denote a graphene sheet with defects in its basal plane, regardless of whether the defects are natively present or intentionally produced. Aside from such apertures, graphene and other two-dimensional materials can represent an impermeable layer to many substances. Therefore, when sized properly, the apertures in the impermeable layer of such materials can be useful for ingress and egress to an enclosure formed from the impermeable layer.

The present disclosure contemplates various graphene-based enclosures that are capable of delivering a target to an in vivo or in vitro location while maintaining a barrier (e.g., an immunoisolation barrier) in an organism or similar biological environment. Encapsulation of molecules or cells with bi-directional transport across a semi-permeable membrane, such as perforated graphene or other two-dimensional materials, while sequestering cells or the like in a biological environment (such as in an organism) can enable treatments to overcome graft rejection, the need for repeated dosages of drugs, and excess surgical intervention. The foregoing can be accomplished by providing technology to allow xenogenic and allogenic tissue transplants, long term low-dose therapeutic levels of a drug, and even sense-response paradigms to treat aliments after surgical intervention, thereby reducing complications from multiple surgeries at the same site. It is to be recognized that the foregoing represent only particular advantages of the present disclosure and should not be considered to limit the scope of the embodiments described herein.

The present inventors recognized that perforated graphene and other two-dimensional materials can readily facilitate the foregoing while surpassing the performance of current delivery vehicles and devices, particularly immune-isolating devices. Graphene can accomplish the foregoing due to its unique thinness, strength, conductivity (for potential electrical stimulation), and permeability in the form of perforations therein. The thinness and subsequent sieve-like transport properties across the graphene membrane surface can allow a disruptive time response to be realized compared to the lengthy diffusion seen with thicker polymeric membranes of comparable size performance.

Two-dimensional materials are, most generally, those which are atomically thin, with thickness from single-layer sub-nanometer thickness to a few nanometers, and which generally have a high surface area. Two-dimensional materials include metal chalogenides (e.g., transition metal dichalogenides), transition metal oxides, hexagonal boron nitride, graphene, silicone and germanene (see: Xu et al. (2013) “Graphene-like Two-Dimensional Materials) Chemical Reviews 113:3766-3798). Graphene represents a form of carbon in which the carbon atoms reside within a single atomically thin sheet or a few layered sheets (e.g., about 20 or less) of fused six-membered rings forming an extended sp2-hybridized carbon planar lattice. In its various forms, graphene has garnered widespread interest for use in a number of applications, primarily due to its favorable combination of high electrical and thermal conductivity values, good in-plane mechanical strength, and unique optical and electronic properties. Other two-dimensional materials having a thickness of a few nanometers or less and an extended planar lattice are also of interest for various applications. In an embodiment, a two dimensional material has a thickness of 0.3 to 1.2 nm. In other embodiment, a two dimensional material has a thickness of 0.3 to 3 nm.

In various embodiments, the two-dimensional material comprises a sheet of a graphene-based material. In an embodiment, the sheet of graphene-based material is a sheet of single or multilayer graphene or a sheet comprising a plurality of interconnected single or multilayer graphene domains. In embodiments, the multilayer graphene domains have 2 to 5 layers or 2 to 10 layers. In an embodiment, the layer comprising the sheet of graphene-based material further comprises non-graphenic carbon-based material located on the surface of the sheet of graphene-based material. In an embodiment, the amount of non-graphenic carbon-based material is less than the amount of graphene. In embodiments, the amount of graphene in the graphene-based material is from 60% to 95% or from 75% to 100%.

In embodiments, the characteristic size of the perforation is from 0.3 to 10 nm, from 1 to 10 nm, from 5 to 10 nm, from 5 to 20 nm, from 10 nm to 50 nm, from 50 nm to 100 nm, from 50 nm to 150 nm, from 100 nm to 200 nm, or from 100 nm to 500 nm. In an embodiment, the average pore size is within the specified range. In embodiments, 70% to 99%, 80% to 99%, 85% to 99% or 90 to 99% of the perforations in a sheet or layer fall within a specified range, but other pores fall outside the specified range.

The technique used for forming the graphene or graphene-based material in the embodiments described herein is not believed to be particularly limited. For example, in some embodiments CVD graphene or graphene-based material can be used. In various embodiments, the CVD graphene or graphene-based material can be liberated from its growth substrate (e.g., Cu) and transferred to a polymer backing. Likewise, the techniques for introducing perforations to the graphene or graphene-based material are also not believed to be particularly limited, other than being chosen to produce perforations within a desired size range. Perforations are sized as described herein to provide desired selective permeability of a species (atom, molecule, protein, virus, cell, etc.) for a given application. Selective permeability relates to the propensity of a porous material or a perforated two-dimensional material to allow passage (or transport) of one or more species more readily or faster than other species. Selective permeability allows separation of species which exhibit different passage or transport rates. In two-dimensional materials selective permeability correlates to the dimension or size (e.g., diameter) of apertures and the relative effective size of the species. Selective permeability of the perforations in two-dimensional materials such as graphene-based materials can also depend on functionalization of perforations (if any) and the specific species that are to be separated. Separation of two or more species in a mixture includes a change in the ratio(s) (weight or molar ratio) of the two or more species in the mixture after passage of the mixture through a perforated two-dimensional material.

Graphene-based materials include, but are not limited to, single layer graphene, multilayer graphene or interconnected single or multilayer graphene domains and combinations thereof. In an embodiment, graphene-based materials also include materials which have been formed by stacking single or multilayer graphene sheets. In embodiments, multilayer graphene includes 2 to 20 layers, 2 to 10 layers or 2 to 5 layers. In embodiments, graphene is the dominant material in a graphene-based material. For example, a graphene-based material comprises at least 30% graphene, or at least 40% graphene, or at least 50% graphene, or at least 60% graphene, or at least 70% graphene, or at least 80% graphene, or at least 90% graphene, or at least 95% graphene. In embodiments, a graphene-based material comprises a range of graphene selected from 30% to 95%, or from 40% to 80% from 50% to 70%, from 60% to 95% or from 75% to 100%.

As used herein, a “domain” refers to a region of a material where atoms are uniformly ordered into a crystal lattice. A domain is uniform within its boundaries, but different from a neighboring region. For example, a single crystalline material has a single domain of ordered atoms. In an embodiment, at least some of the graphene domains are nanocrystals, having domain size from 1 to 100 nm or 10-100 nm. In an embodiment, at least some of the graphene domains have a domain size greater than 100 nm to 1 micron, or from 200 nm to 800 nm, or from 300 nm to 500 nm. “Grain boundaries” formed by crystallographic defects at edges of each domain differentiate between neighboring crystal lattices. In some embodiments, a first crystal lattice may be rotated relative to a second crystal lattice, by rotation about an axis perpendicular to the plane of a sheet, such that the two lattices differ in “crystal lattice orientation”.

In an embodiment, the sheet of graphene-based material comprises a sheet of single or multilayer graphene or a combination thereof. In an embodiment, the sheet of graphene-based material is a sheet of single or multilayer graphene or a combination thereof. In another embodiment, the sheet of graphene-based material is a sheet comprising a plurality of interconnected single or multilayer graphene domains. In an embodiment, the interconnected domains are covalently bonded together to form the sheet. When the domains in a sheet differ in crystal lattice orientation, the sheet is polycrystalline.

In embodiments, the thickness of the sheet of graphene-based material is from 0.34 to 10 nm, from 0.34 to 5 nm, or from 0.34 to 3 nm. In an embodiment, a sheet of graphene-based material comprises intrinsic defects. Intrinsic defects are those resulting from preparation of the graphene-based material in contrast to perforations which are selectively introduced into a sheet of graphene-based material or a sheet of graphene. Such intrinsic defects include, but are not limited to, lattice anomalies, pores, tears, cracks or wrinkles. Lattice anomalies can include, but are not limited to, carbon rings with other than 6 members (e.g. 5, 7 or 9 membered rings), vacancies, interstitial defects (including incorporation of non-carbon atoms in the lattice), and grain boundaries.

In an embodiment, the layer comprising the sheet of graphene-based material further comprises non-graphenic carbon-based material located on the surface of the sheet of graphene-based material. In an embodiment, the non-graphenic carbon-based material does not possess long range order and may be classified as amorphous. In embodiments, the non-graphenic carbon-based material further comprises elements other than carbon and/or hydrocarbons. Non-carbon elements which may be incorporated in the non-graphenic carbon include, but are not limited to, hydrogen, oxygen, silicon, copper and iron. In embodiments, the non-graphenic carbon-based material comprises hydrocarbons. In embodiments, carbon is the dominant material in non-graphenic carbon-based material. For example, a non-graphenic carbon-based material comprises at least 30% carbon, or at least 40% carbon, or at least 50% carbon, or at least 60% carbon, or at least 70% carbon, or at least 80% carbon, or at least 90% carbon, or at least 95% carbon. In embodiments, a non-graphenic carbon-based material comprises a range of carbon selected from 30% to 95%, or from 40% to 80%, or from 50% to 70%.

Such nanomaterials in which pores are intentionally created will be referred to herein as “perforated graphene”, “perforated graphene-based materials” or “perforated two-dimensional materials.” The present disclosure is also directed, in part, to perforated graphene, perforated graphene-based materials and other perforated two-dimensional materials containing a plurality of holes of size (or size range) appropriate for a given enclosure application. The size distribution of holes may be narrow, e.g., limited to a 1-10% deviation in size or a 1-20% deviation in size. In an embodiment, the characteristic dimension of the holes is selected for the application. For circular holes, the characteristic dimension is the diameter of the hole. In embodiments relevant to non-circular pores, the characteristic dimension can be taken as the largest distance spanning the hole, the smallest distance spanning the hole, the average of the largest and smallest distance spanning the hole, or an equivalent diameter based on the in-plane area of the pore. As used herein, perforated graphene-based materials include materials in which non-carbon atoms have been incorporated at the edges of the pores.

In various embodiments, the two-dimensional material comprises graphene, molybdenum sulfide, or boron nitride. In more particular embodiments, the two-dimensional material can be graphene. Graphene according to the embodiments of the present disclosure can include single-layer graphene, multi-layer graphene, or any combination thereof. Other nanomaterials having an extended two-dimensional molecular structure can also constitute the two-dimensional material in the various embodiments of the present disclosure. For example, molybdenum sulfide is a representative chalcogenide having a two-dimensional molecular structure, and other various chalcogenides can constitute the two-dimensional material in the embodiments of the present disclosure. Choice of a suitable two-dimensional material for a particular application can be determined by a number of factors, including the chemical and physical environment into which the graphene or other two-dimensional material is to be terminally deployed. For application in the present invention materials employed in making enclosure are preferably biocompatible or can be made biocompatible.

The process of forming holes in graphene and other two-dimensional materials will be referred to herein as “perforation,” and such nanomaterials will be referred to herein as being “perforated.” In a graphene sheet an interstitial aperture is formed by each six carbon atom ring structure in the sheet and this interstitial aperture is less than one nanometer across. In particular, this interstitial aperture is believed to be about 0.3 nanometers across its longest dimension (the center to center distance between carbon atoms being about 0.28 nm and the aperture being somewhat smaller than this distance). Perforation of sheets comprising two-dimensional network structures typically refers to formation of holes larger than the interstitial apertures in the network structure.

Due to the atomic-level thinness of graphene and other two-dimensional materials, it can be possible to achieve high liquid throughput fluxes during separation or filtration processes, even with holes that are in the ranges of 1-20 nm.

Chemical techniques can be used to create holes in graphene and other two-dimensional materials. Exposure of graphene or another two-dimensional material to ozone or atmospheric pressure plasma (e.g., an oxygen/argon or nitrogen/argon plasma) can effect perforation. Physical techniques, such as ion bombardment, can also be used to remove matter from the planar structure of two-dimensional materials in order to create holes. All such physical or chemical methods can be applied for preparation of perforated two-dimensional for use herein dependent upon the hole sizes or range of hole sizes desired for a given application.

In various embodiments of the present disclosure, the holes produced in the graphene or other two-dimensional material can range from about 0.3 nm to about 50 nm in size. In a more specific embodiment, hole seizes can range from 1 nm to 50 nm. In a more specific embodiment, hole seizes can range from 1 nm to 10 nm. In a more specific embodiment, hole seizes can range from 5 nm to 10 nm. In a more specific embodiment, hole seizes can range from 1 nm to 5 nm. In a more specific embodiment, the holes can range from about 0.5 nm to about 2.5 nm in size. In an additional embodiment, the hole size is from 0.3 to 0.5 nm. In a further embodiment, the hole size is from 0.5 to 10 nm. In an additional embodiment, the hole size is from 5 nm to 20 nm. In a further embodiment, the hole size is from 0.7 nm to 1.2 nm. In an additional embodiment, the hole size is from 10 nm to 50 nm. In embodiments where larger hole sizes are preferred, the hole size is from 50 nm to 100 nm, from 50 nm to 150 nm, or from 100 nm to 200 nm.

The term substance is used generically herein to refer to atoms, molecules, viruses, cells, particles and aggregates thereof. Substances of particular interest are molecules of various size, including biological molecules, such as proteins and nucleic acids. Substances can include pharmaceuticals, drugs, medicaments and therapeutics, which include biologics and small molecule drugs.

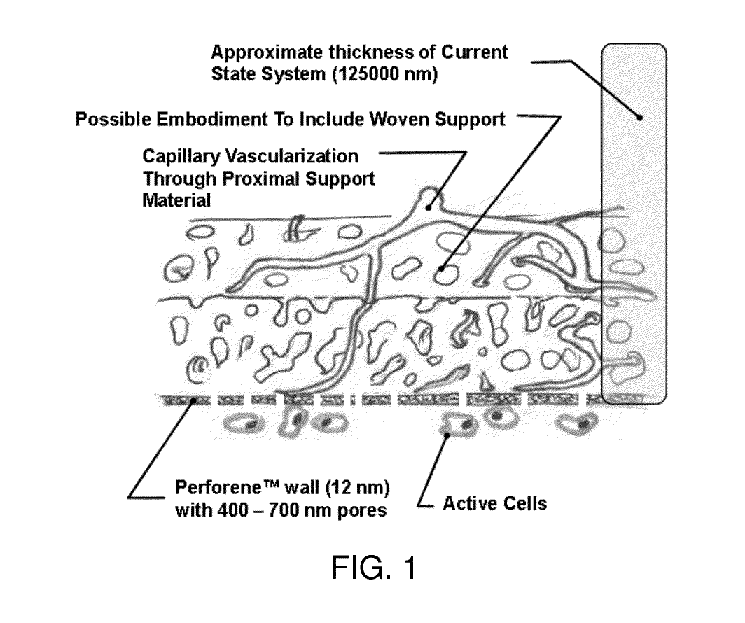

FIG. 1 shows an illustrative schematic demonstrating the thickness of graphene in comparison to conventional drug delivery vehicles and devices. The biocompatibility of graphene can further promote this application, particularly by functionalizing the graphene to be compatible with a particular biological environment (e.g., via available edge bonds, bulk surface functionalization, pi-bonding, and the like). Functionalization can provide membranes having added complexity for use in treating local and systemic disease. FIG. 1 illustrates a wall of an enclosure formed with perforated two-dimensional material having hole sizes in the range of 400-700 nm which will retain active cells. The external biological environment abutting the enclosure (the full enclosure is not shown) is illustrated with an optional porous support structure (polymer or ceramic) adjacent and external to the perforated two-dimensional material and an optional woven support material external to the perforated two-dimensional material. As illustrated, implantation of such an enclosure contemplates vascularization into any such external support materials. In an embodiment intended to provide immunoisolation, generally smaller hole sizes are preferred to prevent entrance of antibodies into the enclosure.

In various embodiments, the present disclosure describes sealed enclosures primarily formed from a two-dimensional material, such as graphene that remain capable of bidirectional transportation of materials. In various embodiments, at least one section or panel of the enclosure contains appropriately sized perforations in the two-dimensional material to allow ingress and egress, respectively, of materials of a desired size to and from the interior of the enclosure.

In some embodiments, the two-dimensional material, such as graphene, can be affixed to a suitable porous substrate. Suitable porous substrates can include, for example, thin film polymers and ceramics.

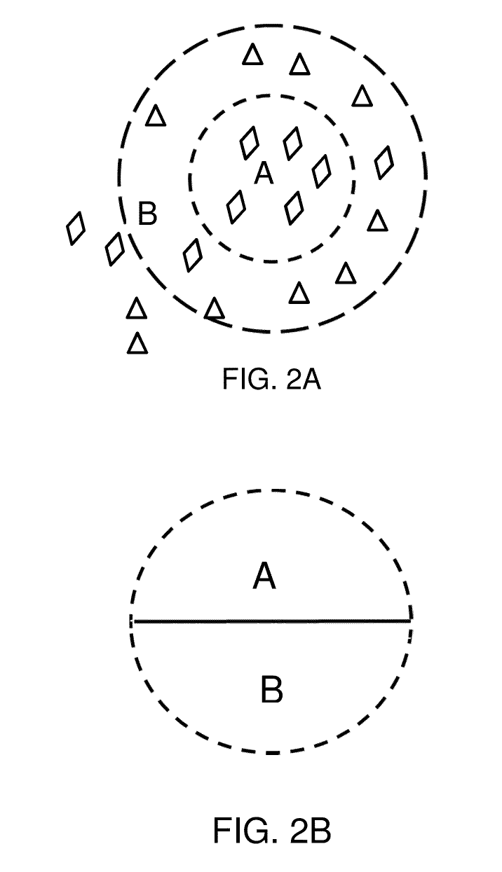

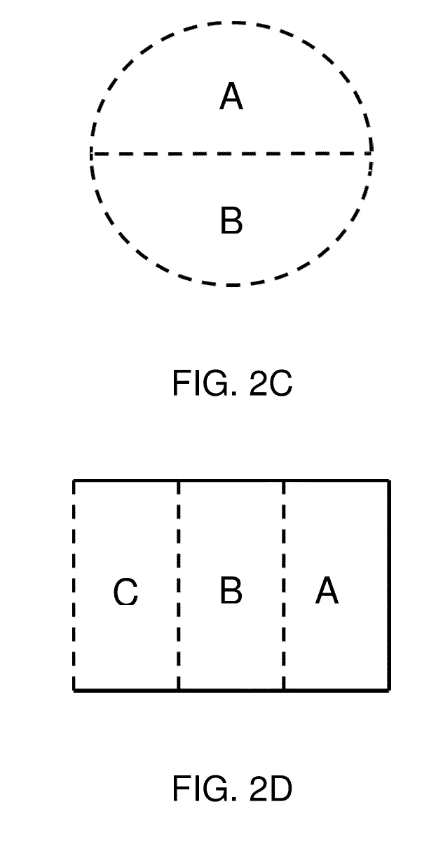

In embodiments, the enclosures can have a plurality of sub-compartments within the main enclosure each sub-compartment comprises perforated two-dimensional material to allow passage of one or more substance into or out of the sub-compartment. In such embodiments, sub-compartment can have any useful shape or size. In specific embodiments, 2 or 3 sub-compartments are present. Several examples of enclosure sub-compartments are illustrated in FIGS. 2A-2D. In FIG. 2A, a nested configuration is illustrated, the main enclosure B completely contains a smaller enclosure A, such that substances in the centermost enclosure A can pass into the main enclosure B, and potentially react with or within the main compartment during ingress and egress therefrom. In this embodiment, one or more substance in A can pass into B and one or more substance in A can be retained in A and not to B. Two sub compartments in which one or more substance can pass directly between the sub-compartments are in direct fluid communication. Passage between sub-compartments and between the enclosure and the external environment is via passage through the holes of a perforated two-dimensional material. The barrier (membrane, i.e. perforated two-dimensional material) between compartment A and B can be permeable to all substances in A or selectively permeable to certain substances in A. The barrier (membrane) between B and the external environment can be permeable to all substances in B or selectively permeable to certain substances in B. In FIG. 2A, sub-compartment A is in direct fluid communication with sub-compartment B which in turn is in direct fluid communication with the external environment. Compartment A in this nested configuration is only in indirect fluid communication with the external environment via intermediate passage into sub-compartment B. The two-dimensional materials employed in different sub-compartments of a given enclosure may be the same or different materials and the perforations or holes sizes in two-dimensional material of different sub-compartments may be the same or different dependent upon the substances involved and the application.

In FIG. 2B the enclosure is bisected with an impermeable wall (e.g., formed of non-porous or non-permeable sealant) forming sub-compartments A and B, such that both sections have access to the egress location independently, but there is no direct or indirect passage of substances from A to B. (It will be appreciated, however, that substances exiting A or B may enter the other sub-compartment indirectly via the external environment.)

In FIG. 2C the main enclosure is again bisected into sub-compartments A and B, but with a perforated material forming the barrier between the sub-compartments. Both sub-compartments not only have access to the egress location independently, but in an embodiment also can interact with one another, i.e. the sub-compartments are in direct fluid communication. In an embodiment, the barrier (membrane) between compartments A and B is selectively permeable, for example allowing at least one substance in A to pass into B, but not allowing the substances originating in B to pass to A.

FIG. 2D illustrates an enclosure having three compartments. The enclosure is illustrated with sub-compartment A having egress into sub-compartment B, which in turn has egress into sub-compartment C, which in turn has egress to the external environment. Compartments A and B have no egress to the external environment, i.e. they are not in direct fluid communication with the external environment. Adjacent sub-compartments A and B and adjacent sub-compartments B and C are each separated by a perforated two-dimensional material and are thus in direct fluid communication with each other. Sub-compartment A is only in indirect fluid communication with compartment C and the external environment via sub-compartment B or B and C, respectively. Various other combinations of semi-permeable barrier (membranes) or non-permeable barriers can be employed to separate compartments in the enclosures herein. Various perforation size constraints can change depending on how the enclosure is ultimately configured (e.g., if one enclosure is within another versus side-by-side). Regardless of the chosen configuration, the boundaries or at least a portion thereof, of the enclosure can be constructed from a two-dimensional material in order to realize the benefits thereof, specifically such that the thickness of the active membrane is less than the diameter of the target to be passed selectively across the membrane. In some embodiments, the pore size of the two-dimensional material can range between about 0.3 nm to about 10 nm in size. Larger pore sizes are also possible.

It should also be noted that in some embodiments, the enclosure can be supported by one or more support structures. In an embodiment, the support structure can itself have a porous structure wherein the pores are larger than those of the two-dimensional material. In an embodiment, the support structure is entirely porous. In embodiments, the support structure is at least in part non-porous.

The multiple physical embodiments for the enclosures and their uses that are described herein can allow for various levels of interaction and scaled complexity of problems to be solved. For example, a single enclosure can provide drug elution for a given time period, or there can be multiple sizes of perforations to restrict or allow movement of distinct targets, each having a particular size.

Added complexity of the embodiments described herein with multiple sub-compartments can allow for interaction between target compounds to catalyze or activate a secondary response (i.e., a “sense-response” paradigm). For example, if there are two sections of an enclosure that have access to egress independently, exemplary compound A may undergo a constant diffusion into the body, or either after time or only in the presence of a stimulus from the body. In such embodiments, exemplary compound A can activate exemplary compound B, or inactivate functionalization blocking exemplary compound B from escaping. The bindings to produce the foregoing effects can be reversible or irreversible. In addition, in other embodiments, exemplary compound A can interact with chemical cascades produced outside the enclosure, and a metabolite subsequent to the interaction can release exemplary compound B (by inactivating functionalization). Further examples utilizing effects that take place in a similar manner include using source cells (non-host, allogenic) contained in an enclosure, within which secretions from the cell can produce a “sense-response” paradigm.

In further embodiments, growth factors can be loaded in the enclosure to encourage vascularization (see FIG. 1). In the foregoing embodiments, cell survival can be far superior as a result of bi-directional transport of nutrients and waste.

In further embodiments, the relative thinness of graphene can enable bi-directional transport across the membrane enclosure in close proximity to blood vessels, particularly capillary blood vessels, and other target cells. The present embodiments using a graphene-based enclosure can provide differentiation over other solutions accomplishing the same effect because the graphene membrane is not appreciably limiting the permeability. Instead, the diffusion of molecules through the medium or interstitial connections can limit the movement of a target.

In regard to the foregoing, any “sense-response” paradigm with graphene is enabled by a superior time response. The biocompatibility of graphene can further enhance this application, with expansion to functionalized graphene membranes for added complexity in treating local and systemic disease with a predicted lower degree of biofouling (due to functionalization or electrification). Additionally, the mechanical stability of graphene can make it suitable to withstand physical stresses and osmotic stresses within the body.

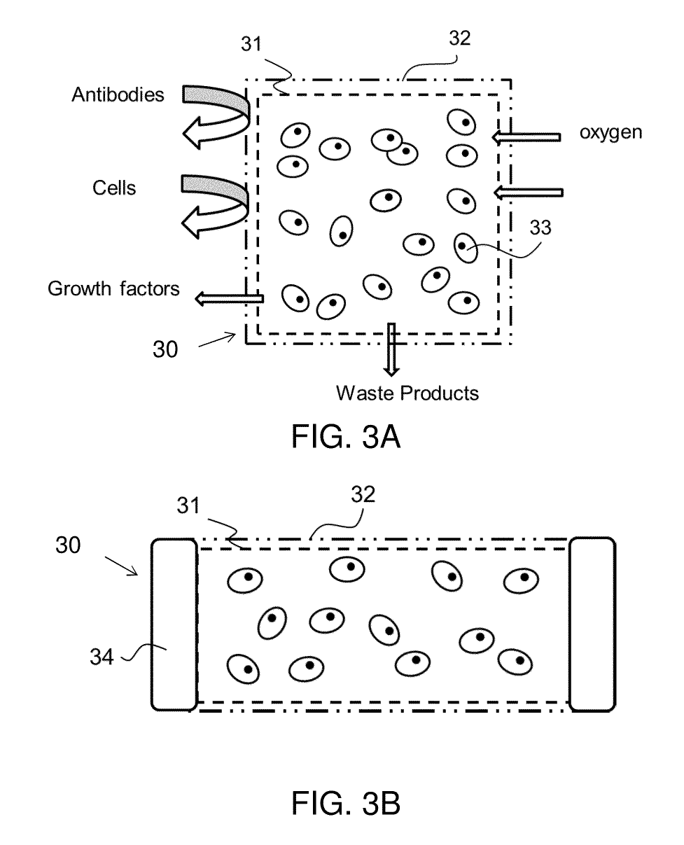

FIGS. 3A and 3B provide a schematic illustration of an enclosure of the invention of immunoisolation. The enclosure is illustrated as having a single compartment. It will be appreciated that the enclosure can having a plurality of sub-compartments, for example, two or three sub-compartments. The enclosure (30) of FIG. 3A is shown in cross-section formed by an inner sheet or layer (31) comprising perforated two-dimensional material, such as a graphene-based material and an outer sheet or layer (32) of a support material. The support material can be porous, selectively permeable or non-porous and non-permeable. However at least a portion of the support material is porous or selectively permeable appropriate for the application of the enclosure. The support sheet or layer can, for example, be a polymer or a ceramic. The enclosure contains selected living cells (33) for a given application. FIG. 3B provides an alternative cross-section of the enclosure of FIG. 3A, showing the space or cavity formed between a first and second composite layer (32/31) where a sealant 34 is illustrated as sealing he edges of the composite layers. It will be appreciated that seals at the edges of the composite layers can be formed employing physical methods of clamping or crimping. Methods and materials for forming the seals at the edges are not particularly limiting but must provide a non-porous and non-permeable seal or closure.

If cells are placed within the closure, at least a portion of the enclosure is permeable to oxygen and nutrients sufficient for cell growth and maintenance and permeable to waste products. The enclosure is not permeable to cells, particularly to immune cells. Cells from the external environment cannot enter the enclosure and cells in the enclosure are retained. The enclosure is not permeable to viruses or bacteria. The enclosure is not permeable to antibodies. In contrast, dependent upon the application, the enclosure is permeable to desirable products, such as growth factors produced by the cells. The cells within the enclosure are immunoisolated. In specific embodiments, hole sizes in perforated two-dimensional materials useful for immunoisolation range in size from 1-10 nm, more preferably 3-10 nm and yet more preferably 3-5 nm.

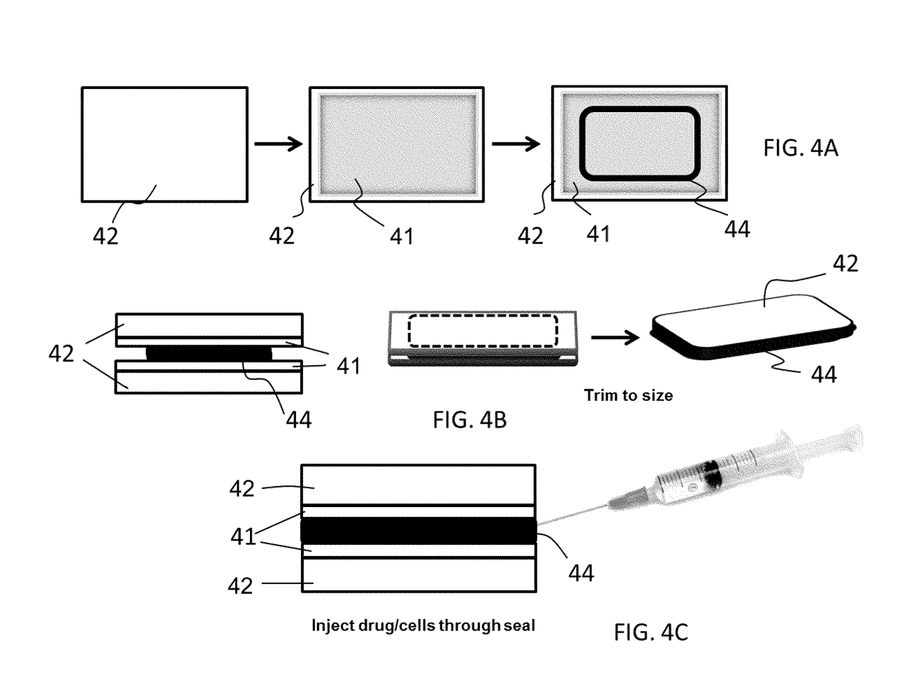

FIGS. 4A-4C illustrate an exemplary method for forming an enclosure of the invention and introducing selected substances, for example cells therein. The method is illustrated with use of a sealant for forming the enclosure. The exemplary enclosure has no sub-compartment. Enclosures with sub-compartments, for example nested or adjacent sub-compartments can be readily prepared employing the illustrated method. As illustrated in FIG. 4A, a first composite layer or sheet is formed by placing a sheet or layer of two-dimensional material, particularly a sheet of graphene-based material or a sheet of graphene (41), in contact with a support layer (42). At least a portion of the support layer (42) of the first composite is porous or permeable. Pore size of the support layer is generally larger than the holes or apertures in the two-dimensional material employed and may be tuned for the environment (e.g. body cavity). A layer of sealant (44), e.g. silicone, is applied on the sheet or layer of perforated two-dimensional material outlining a compartment of the enclosure wherein the sealant will form a non-permeable seal around a perimeter of the enclosure. Formation of a single compartment is illustrate in FIGS. 4A-4C, however, it will be appreciated that multiple independent compartments within an enclosure can be formed by an analogous process. A second composite layer formed in the same way as the first is then prepared and positioned with the sheet or layer of perforated two dimensional materials in contact with the sealant. (Alternatively, a sealant can be applied to a portion of composite layer and the layer can be folded over in contact with the sealant to form an enclosure. A seal is then formed between the two composite layers. Appropriate pressure may be applied to facilitate sealing without damaging the two-dimensional material or its support. It will be appreciated that an alternative enclosure can be formed by applying a sheet or layer of non-porous and non-permeable support material in contact with the sealant. In this case only a portion of the enclosure is porous and permeable. Sealed composite layers are illustrated in FIG. 4B where it is shown that the sealed layers can be trimmed to size around the sealant to form the enclosure. The enclosure formed is shown to have an external porous support layer 42, the sheet or layer of perforated two-dimensional material (41) being positioned as an internal layer, with sealant 44 around the perimeter of the enclosure. As illustrated in FIG. 4C, cells or other substances that would be excluded from passage through the perforated to-dimensional sheet or layer can be introduced into the enclosure after it formed by injection through the sealant layer. Any perforation formed by such injection can be sealed as needed. It will be appreciates that substances and cells can be introduced into the enclosure prior to formation of the seal. Those in the art will appreciate that sterilization methods appropriate for the application envisioned may be employed during or after the preparation of the enclosure.

In an embodiment, the invention provides an enclosure comprising perforated two-dimensional material encapsulating a substance, such that the substance is released to an environment external to the enclosure by passage through the holes in the perforated two-dimensional material. In an embodiment, the enclosure encapsulates more than one different substance. In an embodiment, not all of the different substances are released to an environment external to the enclosure. In an embodiment, all of the different substances are released into an environment external to the enclosure. In an embodiment, different substances are released into an environment external to the enclosure at different rates. In an embodiment, different substances are released into an environment external to the enclosure at the same rates.

In an embodiment, the enclosure comprises two or more sub-compartments, wherein at least one sub-compartment is in direct fluid communication with an environment external to the enclosure through holes in a two-dimensional material of the sub-compartment. In an embodiment, each sub-compartment comprises a perforated two-dimensional material and each sub-compartment is in direct fluid communication with an environment external to the enclosure, through holes in the two-dimensional material of each sub-compartment.

In an embodiment, an enclosure is subdivided into two sub-compartments separated from each other at least in part by perforated two-dimensional material, such that the two-sub-compartments are in direct fluid communication with each other through holes in two-dimensional material. In an embodiment, the enclosure is subdivided into two-sub-compartments each comprising two-dimensional material which sub-compartments are in direct fluid communication with each other through holes in two-dimensional material and only one of the sub-compartments is in direct fluid communication with an environment external to the enclosure. In an embodiment, the enclosure is subdivided into two-sub-compartments each comprising two-dimensional material which sub-compartments are in direct fluid communication with each other through holes in two-dimensional material and both of the sub-compartments are also in direct fluid communication with an environment external to the enclosure.

In an embodiment, the enclosure has an inner sub-compartment and an outer sub-compartment each comprising a perforated two-dimensional material, wherein the inner sub-compartment is entirely enclosed within the outer sub-compartment, the inner and outer compartments are in direct fluid communication with each other through holes in two-dimensional material and the inner sub-compartment is not in direct fluid communication with an environment external to the enclosure.

In an embodiment, where an enclosure has a plurality of sub-compartments each comprising a two-dimensional material, the sub-compartments are nested one within the other, each of which sub-compartments is in direct fluid communication through holes in two-dimensional material with the sub-compartment(s) to which it is adjacent, the outermost sub-compartment in direct fluid communication with an environment external to the enclosure, the remaining plurality of sub-compartments not in direct fluid communication with an environment external to the enclosure.

In an embodiment, where the enclosure is subdivided into a plurality of sub-compartments, each comprising a two-dimensional material, each sub-compartment is in direct fluid communication with one or more adjacent sub-compartments, and only one sub-compartment is in direct fluid communication with an environment external to the enclosure.

In an embodiment of any enclosure configuration herein the at least one substance within the enclosure that is released to an environment external to the enclosure through holes in two-dimensional material is a pharmaceutical, therapeutic or drug. In an embodiment, wherein the released substance is a pharmaceutical, therapeutic or drug, the two-dimensional material of the enclosure for release of the substance comprises holes ranging in size from 1-50 nm. In an embodiment, wherein the released substance is a pharmaceutical, therapeutic or drug, the two-dimensional material of the enclosure for release of the substance comprises holes ranging in size from 1-10 nm.

In an embodiment of any enclosure herein, the substance within the enclosure is cells and the size of the holes in the two-dimensional material is selected to retain the cells within the enclosure and to exclude immune cells and antibodies from entering the enclosure from an environment external to the enclosure. In a specific embodiment, useful for cells, the enclosure is divided into a plurality of sub-compartments and one or more sub-compartments contain cells. An enclosure can contain different cells with a sub-compartment or different cells within different sub-compartments of the same enclosure. In a specific embodiment useful for cells, the enclosure is a nested enclosure wherein the cells are within the inner sub-compartment.

In an embodiment, an enclosure has an inner sub-compartment and an outer sub-compartment each comprising a perforated two-dimensional material wherein the inner sub-compartment is entirely enclosed within the outer sub-compartment, the inner and outer compartments are in direct fluid communication through holes in two-dimensional material of the inner sub-compartment, the inner sub-compartment is not in direct fluid communication with an environment external to the enclosure and the outer compartment is in direct fluid communication with an environment external to the enclosure.

In an embodiment useful with cells, an enclosure has a plurality of sub-compartments each of which comprises perforated two-dimensional material and each of which sub-compartments is in direct fluid communication with one or more adjacent sub-compartments, the cells being within one or more cell-containing sub-compartments each of which are not in direct fluid communication with an environment external to the enclosure.

In embodiments of enclosures containing cells, the cells are yeast cells or bacterial cells. In embodiments of enclosures containing cells, the cells are mammalian cells. In embodiments of enclosures containing cells, the size of the holes, in the two-dimensional material of the enclosure or sub-compartment, ranges from 1-10 nm, 3-10 nm, or from 3-5 nm.

In embodiments of any enclosures herein, two-dimensional material in the enclosure is supported on a porous substrate. In embodiments, the porous substrate can be polymer or ceramic.

In embodiments of any enclosure herein the two-dimensional material is a graphene-based material. In embodiments of any enclosure herein, the two-dimensional material is graphene.

In embodiments of any enclosure herein at least a portion of the holes in the two-dimensional materials of the enclosure are functionalized

In embodiments of any enclosure herein at least a portion of the two-dimensional material is conductive and a voltage can be applied to at least a portion of the conductive two-dimensional material. The voltage can be an AC or DC voltage. The voltage can be applied from a source external to the enclosure. In an embodiment, an enclosure device of the invention further comprises connectors and leads for application of a voltage from an external source to the two-dimensional material.

The invention provides methods employing any enclosure herein in a selected environment for delivery of one or more substance to the environment. In a specific embodiment, the environment is a biological environment. In an embodiment, the enclosure is implanted into biological tissue. In an embodiment, the enclosure is employed for delivery of a pharmaceutical, a drug or a therapeutic.

In an embodiment the invention provides a method comprising introducing an enclosure comprising perforated two-dimensional material into a an environment, the enclosure containing at least one substance; and releasing at least a portion of at least one substance through the holes of the two-dimensional material to the environment external to the enclosure. In an embodiment, the enclosure contains cells which are not released from the enclosure and the at least one substance a portion of which is released is a substance generated by the cells in the enclosure.

In an embodiment the invention provides a method comprising introducing an enclosure comprising perforated two-dimensional material to a environment, the enclosure containing at least one first substance; and migrating a second substance from the environment into the enclosure. In an embodiment, the first substance is cells, a second substance is nutrients and another second substance is oxygen.

In embodiments, the support layer can be a polymer or a ceramic material. Useful exemplary ceramics include nanoporous silica or SiN. Useful porous polymer supports include track-etched polymers, expanded polymers or non-woven polymers. The support material can be porous or permeable. A portion, e.g., a wall, side or portion thereof, of an enclosure or a sub-compartment can be non-porous polymer or ceramic. Biocompatible polymers and ceramics are preferred. A portion of the enclosure can be formed from a sealant, such as a silicone, epoxy, polyurethane or similar material. Biocompatible sealants are preferred.

Additionally, the conductive properties of graphene-based or other two-dimensional membranes can allow for electrification to take place from an external source. In exemplary embodiments, an AC or DC voltage can be applied to conductive two-dimensional materials of the enclosure. The conductivity properties of graphene can provide additional gating to charged molecules. Electrification can occur permanently or only a portion of the time to affect gating. Directional gating of charged molecules can be directed not only through the pores (or restrict travel through pores), but also to the surface of the graphene to adsorb or bind and encourage growth, promote formation of a protective layer, or provide the basis or mechanism for other biochemical effects on the body.

Both permanent and temporary binding to the graphene is possible in such embodiments. In addition to the foregoing advantages, the embodiments described herein can also be advantageous in that they not only represent a disruptive technology for state of the art vehicle and other devices, but they can also permit these vehicles and devices to be used in new ways. For example, cell line developments, therapeutic releasing agents. sensing paradigms (e.g., MRSw's, NMR-based magnetic relaxation switches, see; Koh et al. (2008) Ang. Chem. Int'l Ed. Engl, 47(22)4119-4121) can be used within the enclosures described herein for mitigating biofouling and bioreactivity, conveying superior permeability and less delay in response, and providing mechanical stability. That is, the enclosures described herein can allow existing technologies to be implemented in new ways that are not possible at present.

In addition to the in vivo and in vitro uses described above, the embodiments described herein can be utilized in other areas as well. The enclosures described herein can also be used in non-therapeutic applications such as, for example, the dosage of probiotics in dairy products (as opposed to the presently used microencapsulation techniques to increase viability during processing for delivery to the GI tract). In this regard and others, it should be noted that the enclosures and devices formed therefrom that are described herein can span several orders of magnitude in size, depending on manufacturing techniques and various end use requirements. Nevertheless, the enclosures are believed to be able to be made small enough to circulate through the bloodstream. On the opposite end of the spectrum, the enclosures can be made large enough to implant (on the order of a few inches or greater). These properties can result from the two-dimensional characteristics of the graphene and its growth over large surface areas.

Although the disclosure has been described with reference to the disclosed embodiments, one having ordinary skill in the art will readily appreciate that these are only illustrative of the disclosure. It should be understood that various modifications can be made without departing from the spirit of the disclosure. The disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that aspects of the disclosure may include only some of the described embodiments. Accordingly, the disclosure is not to be seen as limited by the foregoing description.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials and synthetic methods other than those specifically exemplified can be employed in the practice of the invention without resort to undue experimentation. All art-known functional equivalents, of any such methods, device elements, starting materials and synthetic methods are intended to be included in this invention. Whenever a range is given in the specification, for example, a temperature range, a time range, or a composition range, all intermediate ranges and subranges, as well as all individual values included in the ranges given are intended to be included in the disclosure. When a Markush group or other grouping is used herein, all individual members of the group and all combinations and subcombinations possible of the group are intended to be individually included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The preceding definitions are provided to clarify their specific use in the context of the invention.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claims.

1. An enclosure comprising perforated two-dimensional material encapsulating a substance, such that the substance is released to an environment external to the enclosure by passage through the holes in the perforated two-dimensional material.

2. The enclosure of claim 1 encapsulating more than one different substance, wherein not all of the different substances are released to an environment external to the enclosure.

3. (canceled)

4. (canceled)

5. (canceled)

6. The enclosure of claim 1, wherein the enclosure comprises two or more sub-compartments, wherein at least one sub-compartment is in direct fluid communication with an environment external to the enclosure through holes in a two-dimensional material of the sub-compartment.

7. The enclosure of claim 6, wherein each sub-compartment comprises a perforated two-dimensional material and each sub-compartment is in direct fluid communication with an environment external to the enclosure, through holes in the two-dimensional material of each sub-compartment.

8. The enclosure of claim 1, wherein the enclosure is subdivided into two sub-compartments separated from each other at least in part by perforated two-dimensional material, such that the two-sub-compartments are in direct fluid communication with each other through holes in two-dimensional material.

9. The enclosure of claim 1, wherein the enclosure is subdivided into two-sub-compartments each comprising two-dimensional material which sub-compartments are in direct fluid communication with each other through holes in two-dimensional material and only one of the sub-compartments is in direct fluid communication with an environment external to the enclosure.

10. The enclosure of claim 1, wherein the enclosure is subdivided into two-sub-compartments each comprising two-dimensional material which sub-compartments are in direct fluid communication with each other through holes in two-dimensional material and both of the sub-compartments are also in direct fluid communication with an environment external to the enclosure.

11. The enclosure of claim 1 having an inner sub-compartment and an outer sub-compartment each comprising a perforated two-dimensional material, wherein the inner sub-compartment is entirely enclosed within the outer sub-compartment, the inner and outer compartments are in direct fluid communication with each other through holes in two-dimensional material and the inner sub-compartment is not in direct fluid communication with an environment external to the enclosure.

12. The enclosure of claim 1 having a plurality of sub-compartments each comprising a two-dimensional material, the sub-compartments nested one within the other, each of which sub-compartments is in direct fluid communication through holes in two-dimensional material with the sub-compartment(s) to which it is adjacent, the outermost sub-compartment in direct fluid communication with an environment external to the enclosure, the remaining plurality of sub-compartments not in direct fluid communication with an environment external to the enclosure.

13. The enclosure of claim 1 subdivided into a plurality of sub-compartment, each comprising a two-dimensional material, wherein each sub-compartment is in direct fluid communication with one or more adjacent sub-compartments, but wherein only one sub-compartment is in direct fluid communication with an environment external to the enclosure.

14. (canceled)

15. (canceled)

16. (canceled)

17. The enclosure of claim 1, wherein a substance within the enclosure is cells and the size of the holes in the two-dimensional material is selected to retain the cells within the enclosure and to exclude immune cells and antibodies from entering the enclosure from an environment external to the enclosure.

18. (canceled)

19. The enclosure of claim 17 having an inner sub-compartment and an outer sub-compartment each comprising a perforated two-dimensional material wherein the inner sub-compartment is entirely enclosed within the outer sub-compartment, the inner and outer compartments are in direct fluid communication through holes in two-dimensional material of the inner sub-compartment, the inner sub-compartment is not in direct fluid communication with an environment external to the enclosure and the outer compartment is in direct fluid communication with an environment external to the enclosure.

20. The enclosure of claim 17 having a plurality of sub-compartments each of which comprises perforated two-dimensional material and each of which sub-compartments is in direct fluid communication with one or more adjacent sub-compartments, the cells being within one or more cell-containing sub-compartments each of which are not in direct fluid communication with an environment external to the enclosure.

21. (canceled)

22. (canceled)

23. (canceled)

24. (canceled)

25. The enclosure of claim 1, wherein the two-dimensional material is supported on a porous substrate.

26. (canceled)

27. The enclosure claim 1, wherein the two-dimensional material is a graphene-based material.

28. The enclosure of claim 1, wherein at least a portion of the holes in the two-dimensional material are functionalized, at least a portion of the two-dimensional material is conductive or both.

29. (canceled)

30. A method comprising:
introducing an enclosure comprising perforated two-dimensional material to a an environment, the enclosure containing at least one substance; and
releasing at least a portion of at least one substance through the holes of the two-dimensional material to the environment external to the enclosure.

31. (canceled)

32. (canceled)

33. (canceled)

34. A method comprising:
introducing an enclosure of claim 1 into an environment, the enclosure containing at least one substance; and
releasing at least a portion of at least one substance through the holes of the two-dimensional material to the environment external to the enclosure.

35. A method comprising:
introducing an enclosure of claim 17 into an environment; and
releasing at least a portion of at least one substance through the holes of the two-dimensional material to the environment external to the enclosure wherein the at least one substance is a substance generated by the cells within the enclosure.

36. A method comprising:
introducing an enclosure comprising perforated two-dimensional material to a environment, the enclosure containing at least one first substance; and
migrating a second substance from the environment into the enclosure.

37. The method of claim 36, wherein the first substance is cells, a second substance is nutrients and another second substance is oxygen.
